(19) United States
(12) Patent Application Publication
(10) Pub. No.: US 2026/0102130 A1
(43) Pub. Date: Apr. 16, 2026

(54) EXAMINING TABLE FOR BONE MINERAL DENSITY MEASUREMENT

(71) Applicant: Northern Jiangsu People's Hospital, Yangzhou (CN)

(72) Inventor: Xia Lu, Yangzhou (CN)

(21) Appl. No.: 19/242,943

(22) Filed: Jun. 18, 2025

(30) Foreign Application Priority Data
Oct. 14, 2024 (CN) 202411426698.3

(51) Int. Cl. A61B 6/04; A61B 6/50

(52) U.S. Cl. CPC A61B 6/0421; A61B 6/505

(57) ABSTRACT

Disclosed is an examining table for bone mineral density (BMD) measurement, falling within the field of medical devices. The table includes a table body, a guide block, inclined plates, fixing mechanisms and a drive mechanism. The arranged drive mechanism drives two inclined plates to move synchronously along extension lines of a V-shaped included angle in a same direction, allowing the present disclosure to adjust positions of the inclined plates based on the placement of a patient's feet. When used in conjunction with the fixing mechanisms on the inclined plates, patients of different heights may always maintain a standardized posture during hip joint BMD measurement, ensuring accuracy of measurement results.

This application claims priority of Chinese Patent Application No. 202411426698.3, filed on October 14, 2024, the entire contents of which are incorporated herein by reference.

### TECHNICAL FIELD

The present disclosure relates to the field of medical devices, and in particular to an examining table for bone mineral density (BMD) measurement.

### BACKGROUND

Dual-energy X-ray absorptiometry (DXA) is considered a gold standard for osteoporosis diagnosis in populations, typically measuring BMD values at L1-L4 lumbar vertebrae and a femoral neck of a hip joint region.

Osteoporosis predominantly affects older adults, and hyperosteogeny at margins of lumbar vertebrae is often observed in this population, which may interfere with BMD diagnosis. Therefore, in cases where lumbar vertebrae are accompanied by hyperosteogeny, the measurement of BMD at the hip joint becomes particularly important.

During hip joint BMD measurement, adequate exposure of the femoral neck at the hip joint region is required. Therefore, a patient needs to maintain a standardized posture (supine posture with precise leg abduction and internal rotation angles) in the process of diagnosis, serving as an essential prerequisite for ensuring accuracy of BMD measurement results.

In the related art, an isosceles trapezoidal frame is typically arranged on an examining table, with the patient's feet fixed to two side surfaces of the isosceles trapezoidal frame using straps, thereby ensuring that the patient's posture remains stationary during the examination. However, for patients of different heights, the isosceles trapezoidal frame is usually fixed to the examining table, resulting in inconsistent leg abduction angles when the patient's feet are fixed to the isosceles trapezoidal frame. Therefore, the above method cannot guarantee the accuracy of the measurement results for patients of different heights.

### SUMMARY

An objective of the present disclosure is to provide an examining table for BMD measurement to solve the problems existing in above the related art.

The present disclosure employs the following technical solutions.

The present disclosure provides an examining table for BMD measurement, including a table body;

a guide block, arranged at a middle part of a top face of the table body, a V-shaped included angle being formed by two side edges of the guide block;

two inclined plates, oppositely arranged and located on extension lines of the V-shaped included angle;

fixing mechanisms, arranged on the inclined plates and used for fixing patient's feet; and

a drive mechanism, arranged on the table body and used for driving the two inclined plates to move synchronously along the extension lines of the V-shaped included angle in a same direction.

In a further description, each fixing mechanism includes an arc-shaped groove movably arranged on an outer side of the inclined plate, an airbag is arranged in the arc-shaped groove, and the airbag is connected to an inflation and deflation assembly; and an adjustment assembly is arranged on the inclined plate, and an operating end of the adjustment assembly is connected to the arc-shaped groove.

In a further description, the adjustment assembly includes linear drive components arranged on an inner side of the inclined plate, operating ends of the linear drive components are connected to a connecting piece, and two ends of the connecting piece pass through long holes on two sides of the inclined plate and are connected to the arc-shaped groove.

In a further description, the inflation and deflation assembly includes an intake pipe and an exhaust pipe, the intake pipe and the exhaust pipe pass through avoidance holes on the arc-shaped groove and are connected to the airbag, and an inflation valve and a deflation valve are arranged on the intake pipe and the exhaust pipe; and a hose is connected to the intake pipe, and a tail end of the hose is connected to an air pump.

In a further description, the drive mechanism includes two sliding plates arranged oppositely, the two sliding plates are slidably connected to sliding holes on the top face of the table body, and the two sliding holes are located on the extension lines of the V-shaped included angle; tops of the sliding plates are connected to the corresponding inclined plates, with bottoms penetrating through the sliding holes and being connected to movable blocks Ⅰ; and guide rods movably penetrate through the movable blocks Ⅰ, the two guide rods are arranged in a same direction as the sliding holes, and two ends of the guide rods are connected to the table body; and

a movable block Ⅱ is arranged between the two movable blocks Ⅰ, the movable block Ⅱ is connected to a straight-line reciprocating assembly, and the straight-line reciprocating assembly drives the movable block Ⅱ to move along a lengthwise direction of the table body; and a connecting rod is connected to the movable block Ⅱ, two ends of the connecting rod movably penetrate through the corresponding movable blocks Ⅰ, and an axial direction of the connecting rod is perpendicular to a movement direction of the movable block Ⅱ.

In a further description, the straight-line reciprocating assembly includes a threaded rod rotatably arranged between the two guide rods, the movable block Ⅱ is threadedly sleeved on the threaded rod, and an axial direction of the threaded rod is the same as the lengthwise direction of the table body; and a rotating drive component is arranged on the table body through a mounting seat, and the rotating drive component is transmission-connected to the threaded rod through a transmission portion.

In a further description, the transmission portion includes a worm dynamically connected to an output shaft of the rotating drive component, one side of the worm is meshed to a worm gear, and the worm gear is sleeved on the threaded rod.

In a further description, two ends of the worm are rotatably connected to shaft plates, and the shaft plates are connected to the table body.

In a further description, limit plates are arranged at two ends of the connecting rod.

Compared to the related art, the present disclosure has the following beneficial effects.

The arranged drive mechanism drives the two inclined plates to move synchronously along the extension lines of the V-shaped included angle in a same direction, allowing the present disclosure to adjust positions of the inclined plates based on the placement of the patient's feet. When used in conjunction with the fixing mechanisms on the inclined plates, patients of different heights may always maintain the standardized posture during hip joint BMD measurement, ensuring accuracy of measurement results.

### BRIEF DESCRIPTION OF THE DRAWINGS

The present disclosure is further described below in combination with the accompanying drawings.

FIG. 1 is a perspective schematic structural diagram of an examining table for BMD measurement of the present disclosure;

FIG. 2 is a perspective schematic structural diagram of a fixing mechanism of the present disclosure;

FIG. 3 is a perspective schematic structural diagram of an inflation and deflation assembly of the present disclosure;

FIG. 4 is a perspective schematic structural diagram of a drive mechanism of the present disclosure;

FIG. 5 is a perspective schematic structural diagram of a transmission portion of the present disclosure; and

FIG. 6 is a schematic diagram showing an included angle between inclined plates and a top face of a table body of the present disclosure.

Reference numerals and denotations thereof: 1-table body; 2-guide block; 3-inclined plate; 301-long hole; 4-fixing mechanism; 41-arc-shaped groove; 411-avoidance hole; 42-airbag; 43-inflation and deflation assembly; 431-intake pipe; 432-exhaust pipe; 433-inflation valve; 434-deflation valve; 435-hose; 436-air pump; 44-adjustment assembly; 441-linear drive component; 442-connecting piece; 5-drive mechanism; 51-sliding plate; 52-sliding hole; 53-movable block Ⅰ; 54-guide rod; 55-mounting plate; 56-movable block Ⅱ; 57-straight-line reciprocating assembly; 571-threaded rod; 572-mounting seat; 573-rotating drive component; 574-transmission portion; 5741-worm; 5742-worm gear; 5743-shaft plate; 58-connecting rod; 59-limit plate; 6-connecting plate; and 7-scanning arm.

### DETAILED DESCRIPTION

In order to make the technical problems to be solved, technical solutions and beneficial effects of the present disclosure more obvious, the present disclosure is further described in detail in combination with the accompanying drawings and embodiment.

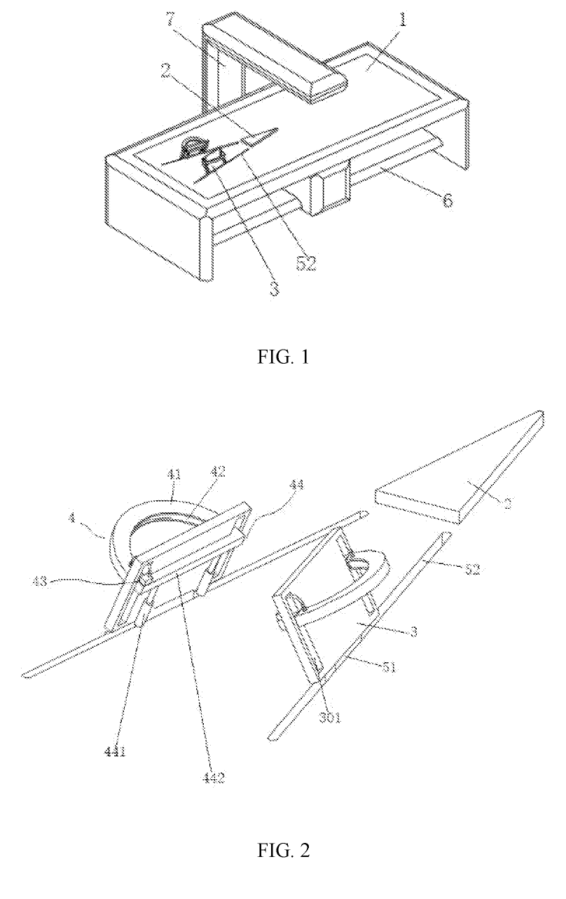

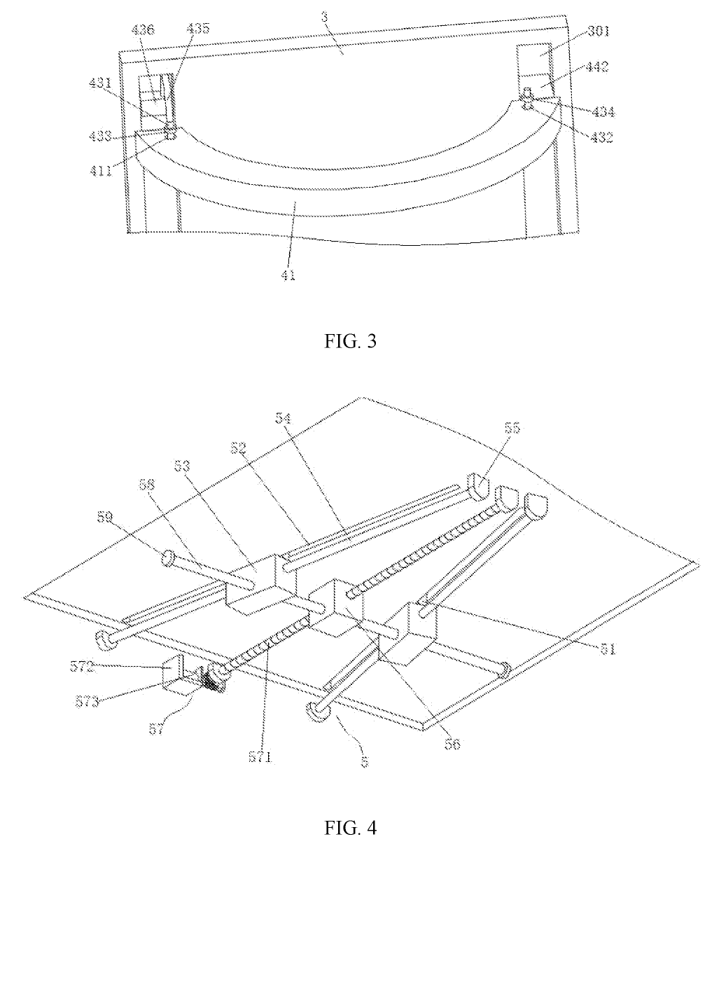

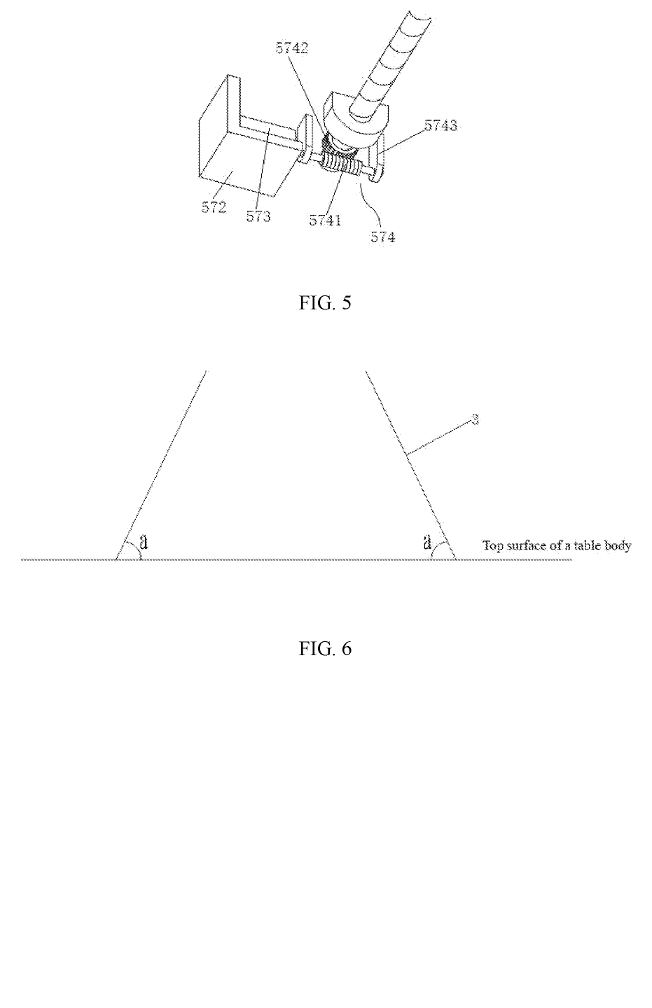

Referring to FIGS. 1-6, the embodiment provides an examining table for BMD measurement, including a table body 1. A guide block 2 is fixed at a middle part of a top face of the table body 1, and a V-shaped included angle is formed by two side edges of the guide block 2. The quantity of inclined plates 3 is two, with the inclined plates 3 being oppositely arranged, and the two inclined plates 3 are located on extension lines of the V-shaped included angle. Fixing mechanisms 4 are arranged on the inclined plates 3, and the fixing mechanisms 4 are used for fixing a patient's feet. A drive mechanism 5 is arranged on the table body 1, and the drive mechanism 5 is used for driving the two inclined plates 3 to move synchronously along the extension lines of the V-shaped included angle in a same direction.

In this embodiment, the V-shaped included angle corresponds to a leg abduction angle required for hip joint BMD measurement. With patient's feet fixed to outer surfaces of the inclined plates 3 through the fixing mechanisms 4, an internal rotation angle of both legs is an internal rotation angle in a standardized posture for hip joint BMD measurement at this time.

Specifically, an included angle a between each inclined plate 3 and the top face of the table body 1 can be set within 60°-70°. In the embodiment, the included angle a is set at 65°.

When in use of the present disclosure, the patient assumes a supine position on the top face of the table body 1 with both legs abducted along the two side edges of the guide block 2. Due to variations in leg length among patients of different heights, the drive mechanism 5 is arranged in the present disclosure. The arranged drive mechanism 5 drives the two inclined plates 3 to move synchronously along the extension lines of the V-shaped included angle in a same direction, allowing the present disclosure to adjust positions of the inclined plates 3 based on the placement of the patient's feet. When used in conjunction with the fixing mechanisms 4 on the inclined plates 3, the patients of different heights can always maintain the standardized posture during hip joint BMD measurement, thereby ensuring accuracy of measurement results.

In a further optimization solution, each fixing mechanism 4 includes an arc-shaped groove 41 movably arranged on an outer side of the inclined plate 3, an airbag 42 is arranged in the arc-shaped groove 41, and the airbag 42 is connected to an inflation and deflation assembly 43; and an adjustment assembly 44 is arranged on the inclined plate 3, and an operating end of the adjustment assembly 44 is connected to the arc-shaped groove 41.

In this embodiment, the adjustment assemblies 44 are used for driving the arc-shaped grooves 41 to move in a straight line along the outer surfaces of the inclined plates 3, allowing the patient's feet to be positioned within areas between the arc-shaped grooves 41 and the inclined plates 3. After the airbags 42 are inflated through the inflation and deflation assemblies 43, the airbags 42 are in an expanded state, thereby fixing patient's feet through the expanded airbags 42.

In a further optimization solution, the adjustment assembly 44 includes linear drive components 441 arranged on an inner side of the inclined plate 3, operating ends of the linear drive components 441 are connected to a connecting piece 442, and two ends of the connecting piece 442 pass through long holes 301 on two sides of the inclined plate 3 and are connected to the arc-shaped groove 41.

In this embodiment, each linear drive component 441 is configured as an electric push rod, with a fixing end of the linear drive component 441 fixedly connected to an inner surface of the inclined plate 3. Each connecting piece 442 is configured as a gantry-type structure, two ends of the connecting piece 442 are fixedly connected to two ends of the arc-shaped groove 41, and the two ends of the connecting piece 442 are slidably connected inside the long holes 301. The arranged linear drive components 441 drives the connecting pieces 442 to move, thereby driving the arc-shaped grooves 41 and the airbags 42 as a whole to move in a straight line along the outer surfaces of the inclined plates 3.

In a further optimization solution, the inflation and deflation assembly 43 includes an intake pipe 431 and an exhaust pipe 432, the intake pipe 431 and the exhaust pipe 432 pass through avoidance holes 411 on the arc-shaped groove 41 and are connected to the airbag 42, and an inflation valve 433 and a deflation valve 434 are fixedly mounted on the intake pipe 431 and the exhaust pipe 432; and a hose 435 is connected to the intake pipe 431, and a tail end of the hose 435 is connected to an air pump 436.

In this embodiment, the air pumps 436 are fixedly mounted on the connecting pieces 442, and output ends of the air pumps 436 are fixedly communicated with the intake pipes 431 through the hoses 435; the intake pipes 431 and the exhaust pipes 432 are located on two sides of the arc-shaped grooves 41, and bottoms of the intake pipes 431 and the exhaust pipes 432 are fixedly communicated with two ends of the airbags 42; and the hoses 435 and the avoidance holes 411 can all accommodate the movement requirements of the intake pipes 431 and the exhaust pipes 432 during inflation and deflation cycles of the airbags 42.

By employing this solution, when it is necessary to fix the patient's feet, the inflation valves 433 are opened and the deflation valves 434 are closed, and the air pumps 436 are started to inflate the airbags 42. After inflation, and the airbags 42 will expand upon inflation. After the patient's feet have been fixed through the airbags 42, the inflation valves 433 are closed, causing the air pumps 436 to stop. Upon completion of the BMD measurement, the deflation valves 434 are opened to release the gas from within the airbags 42. After the airbags 42 are reduced in size, the fixation applied to the patient's feet can be released. Furthermore, the linear drive components 441 drive the connecting pieces 442 to move, thereby causing the patient's feet to be withdrawn from the areas enclosed by the arc-shaped grooves 41 and the inclined plates 3.

In a further optimization description, the drive mechanism 5 includes two sliding plates 51 arranged oppositely, the two sliding plates 51 are slidably connected to sliding holes 52 on the top face of the table body 1, and the two sliding holes 52 are located on the extension lines of the V-shaped included angle; tops of the sliding plates 51 are connected to the corresponding inclined plates 3, with bottoms of the sliding plates 51 penetrating through the sliding holes 52 and being fixedly connected to movable blocks Ⅰ 53; and guide rods 54 movably penetrate through the movable blocks Ⅰ 53, the two guide rods 54 are arranged in a same direction as the sliding holes 52, and two ends of the guide rods 54 are fixedly connected to the table body 1 through mounting plates 55; and

a movable block Ⅱ 56 is arranged between the two movable blocks Ⅰ 53, the movable block Ⅱ 56 is connected to a straight-line reciprocating assembly 57, and the straight-line reciprocating assembly 57 drives the movable block Ⅱ 56 to move along a lengthwise direction of the table body 1; and a connecting rod 58 is connected to the movable block Ⅱ 56, two ends of the connecting rod 58 movably penetrate through the corresponding movable blocks Ⅰ 53, and an axial direction of the connecting rod 58 is perpendicular to a movement direction of the movable block Ⅱ 56.

In this embodiment, through holes are arranged on the movable block Ⅱ 56 and the movable blocks Ⅰ 53, and the connecting rod 58 horizontally penetrates through the through holes of the movable block Ⅱ 56 and the movable blocks Ⅰ 53, thereby forming a fixing connection between the connecting rod 58 and the movable block Ⅱ 56 while maintaining a sliding connection between the connecting rod 58 and the movable blocks Ⅰ 53. Moreover, the two ends of the connecting rod 58 are fixedly connected to limit plates 59, and the arranged limit plates 59 can effectively prevent the connecting rod 58 from disengaging from the through holes on the movable blocks Ⅰ 53.

By employing this solution, the arranged straight-line reciprocating assembly 57 drives the movable block Ⅱ 56 to perform rectilinear motion along a lengthwise direction of the table body 1. Under the joint action of the connecting rod 58 and the guide rods 54, the two movable blocks Ⅰ 53 can synchronously move along axial directions of the guide rods 54 in the same direction, thereby effecting synchronous co-directional displacement of the two inclined plates 3 along the extension lines of the V-shaped included angle under the action of the sliding plates 51 and the sliding holes 52.

In a further optimization description, the straight-line reciprocating assembly 57 includes a threaded rod 571 rotatably arranged between the two guide rods 54, the movable block Ⅱ 56 is threadedly sleeved on the threaded rod 571, and an axial direction of the threaded rod 571 is the same as the lengthwise direction of the table body 1; and a rotating drive component 573 is arranged on the table body 1 through a mounting seat 572, and the rotating drive component 573 is transmission-connected to the threaded rod 571 through a transmission portion 574.

In this embodiment, the rotating drive component 573 is implemented as a servo motor, a fixing end of the rotating drive component 573 is fixed to the mounting seat 572, and the mounting seat 572 is fixed to the table body 1. Through the arranged rotating drive component 573, the threaded rod 571 rotates clockwise or counterclockwise under the action of the transmission portion 574, thereby driving the movable block Ⅱ 56 to perform straight-line reciprocating motion along the axial direction of the threaded rod 571.

In a further optimization solution, the transmission portion 574 includes a worm 5741 dynamically connected to an output shaft of the rotating drive component 573, one side of the worm 5741 is meshed to a worm gear 5742, and the worm gear 5742 is fixedly sleeved on the threaded rod 571.

In this embodiment, two ends of the worm 5741 are rotatably connected to shaft plates 5743, and the shaft plates 5743 are fixedly connected to the table body 1. The rotating drive component 573 drives the worm 5741 to rotate, and the worm 5741 drives the worm gear 5742 to rotate, thereby driving the threaded rod 571 to rotate. By utilizing the self-locking property inherent in the meshing connection between the worm gear 5742 and the worm 5741, the stability of the movable block Ⅱ 56 can be maintained after position adjustment, thereby ensuring the stability of the movable blocks Ⅰ 53, the sliding plates 51, and the inclined plates 3.

In a further optimization solution, a horizontally arranged connecting plate 6 is fixed between two side walls of the table body 1, and the connecting plate 6 is located below the top surface of the table body 1. In this embodiment, the connecting plate 6 is used for mounting a dual-energy X-ray scanning arm 7.

It is to be noted that the scanning arm 7 is the related art. Its operating principle and application method are well-established and are not be elaborated herein.

The foregoing embodiments are merely to describe preferred implementations of the present disclosure, rather than limiting the scope of the present disclosure. Without departing from the design spirit of the present disclosure, any modifications or improvements to the technical solutions of the present disclosure made by a person skilled in the art shall fall within the scope of protection defined by the claims of the present disclosure.

1. An examining table for bone mineral density (BMD) measurement, comprising a table body (1);a guide block (2), arranged at a middle part of a top face of the table body (1), a V-shaped included angle being formed by two side edges of the guide block (2);two inclined plates (3), oppositely arranged and located on extension lines of the V-shaped included angle;fixing mechanisms (4), arranged on the inclined plates (3) and used for fixing patient's feet; and a drive mechanism (5), arranged on the table body (1) and used for driving the two inclined plates (3) to move synchronously along the extension lines of the V-shaped included angle in a same direction.

2. The examining table for BMD measurement according to claim 1, wherein each fixing mechanism (4) comprises an arc-shaped groove (41) movably arranged on an outer side of the inclined plate (3), an airbag (42) is arranged in the arc-shaped groove (41), and the airbag (42) is connected to an inflation and deflation assembly (43); and an adjustment assembly (44) is arranged on the inclined plate (3), and an operating end of the adjustment assembly (44) is connected to the arc-shaped groove (41).

3. The examining table for BMD measurement according to claim 2, wherein the adjustment assembly (44) comprises linear drive components (441) arranged on an inner side of the inclined plate (3), operating ends of the linear drive components (441) are connected to a connecting piece (442), and two ends of the connecting piece (442) pass through long holes (301) on two sides of the inclined plate (3) and are connected to the arc-shaped groove (41).

4. The examining table for BMD measurement according to claim 2, wherein the inflation and deflation assembly (43) comprises an intake pipe (431) and an exhaust pipe (432), the intake pipe (431) and the exhaust pipe (432) pass through avoidance holes (411) on the arc-shaped groove (41) and are connected to the airbag (42), and an inflation valve (433) and a deflation valve (434) are arranged on the intake pipe (431) and the exhaust pipe (432); and a hose (435) is connected to the intake pipe (431), and a tail end of the hose (435) is connected to an air pump (436).

5. The examining table for BMD measurement according to claim 1, wherein the drive mechanism (5) comprises two sliding plates (51) arranged oppositely, the two sliding plates (51) are slidably connected to sliding holes (52) on the top face of the table body (1), and the two sliding holes (52) are located on the extension lines of the V-shaped included angle; tops of the sliding plates (51) are connected to the corresponding inclined plates (3), with bottoms penetrating through the sliding holes (52) and being connected to movable blocks Ⅰ (53); and guide rods (54) movably penetrate through the movable blocks Ⅰ (53), the two guide rods (54) are arranged in a same direction as the sliding holes (52), and two ends of the guide rods are connected to the table body (1); and
a movable block Ⅱ (56) is arranged between the two movable blocks Ⅰ (53), the movable block Ⅱ (56) is connected to a straight-line reciprocating assembly (57), and the straight-line reciprocating assembly (57) drives the movable block Ⅱ (56) to move along a lengthwise direction of the table body (1); and a connecting rod (58) is connected to the movable block Ⅱ (56), two ends of the connecting rod (58) movably penetrate through the corresponding movable blocks Ⅰ (53), and an axial direction of the connecting rod (58) is perpendicular to a movement direction of the movable block Ⅱ (56).

6. The examining table for BMD measurement according to claim 5, wherein the straight-line reciprocating assembly (57) comprises a threaded rod (571) rotatably arranged between the two guide rods (54), the movable block Ⅱ (56) is threadedly sleeved on the threaded rod (571), and an axial direction of the threaded rod (571) is the same as the lengthwise direction of the table body (1); and a rotating drive component (573) is arranged on the table body (1) through a mounting seat (572), and the rotating drive component (573) is transmission-connected to the threaded rod (571) through a transmission portion (574).

7. The examining table for BMD measurement according to claim 6, wherein the transmission portion (574) comprises a worm (5741) dynamically connected to an output shaft of the rotating drive component (573), one side of the worm (5741) is meshed to a worm gear (5742), and the worm gear (5742) is sleeved on the threaded rod (571).

8. The examining table for BMD measurement according to claim 7, wherein two ends of the worm (5741) are rotatably connected to shaft plates (5743), and the shaft plates (5743) are connected to the table body (1).

9. The examining table for BMD measurement according to claim 5, wherein limit plates (59) are arranged at two ends of the connecting rod (58).
